(19) United States
(12) Patent Application Publication
(10) Pub. No.: US 2020/0315518 A1
(43) Pub. Date: Oct. 8, 2020

(54) APPARATUS FOR PROCESSING DATA FOR PREDICTING DEMENTIA THROUGH MACHINE LEARNING, METHOD THEREOF, AND RECORDING MEDIUM STORING THE SAME

(71) Applicant: KOREA INSTITUTE OF SCIENCE & TECHNOLOGY INFORMATION, Daejeon (KR)

(72) Inventors: Hong Woo Chun, Seoul (KR); Hee Chel Kim, Seoul (KR); Seon Ho Kim, Gyeonggi-do (KR); Jung Joon Kim, Seoul (KR); Byoung Youl Coh, Seoul (KR); Oh Jin Kwon, Seoul (KR); Yeong Ho Moon, Seoul (KR)

(21) Appl. No.: 16/908,563

(22) Filed: Jun. 22, 2020

Related U.S. Application Data
(63) Continuation of application No. PCT/KR2019/002350, filed on Feb. 27, 2019.

(30) Foreign Application Priority Data
Feb. 27, 2018 (KR) 10-2018-0023585

(51) Int. Cl. A61B 5/00; A61B 5/145; G16H 20/70; G16H 50/20; G16H 50/30; G16H 50/70

(52) U.S. Cl. CPC A61B 5/4088; A61B 5/7275; A61B 5/7264; G16H 50/70; G16H 20/70; G16H 50/20; G16H 50/30; A61B 5/14546

(57) ABSTRACT

The present disclosure processes a user's medical data for each year to be input to a machine learning device for predicting dementia, and a data set composed of optimal features is constructed. The optimal features include at least information on the user's disease history, and the user's medical information for each year in the last 7 years or less. Precise prediction and diagnosis of dementia may be made by constructing the optimal features identified through experiments in the user's medical data for each year. Since the experimental results show that the prediction results of observing a disease history of 7 years or less may be the best, rather than observing medical information for a long period of time, the appropriate criteria may be suggested for predicting dementia.

### CROSS-REFERENCE TO RELATED APPLICATION

This application is a Continuation of International Application No. PCT/KR2019/002350 filed Feb. 27, 2019, which claims benefit of priority to Korean Patent Application No. 10-2018-0023585 filed Feb. 27, 2018, the entire content of which is incorporated herein by reference.

### BACKGROUND

### 1. Technical Field

The present disclosure relates to processing data for predicting dementia, and more specifically, to an apparatus and a method for processing medical data of a user to be input to a machine learning device in order to predict a user's dementia through machine learning.

### 2. Description of the Related Art

Research on the treatment of dementia has been conducted worldwide for over 20 years, but it is a symptom that has not yet been fully cured.

Dementia, one of the senile diseases, has increased rapidly with the global increase in senior population. In the United States, the mortality rate from Alzheimer's dementia doubled between 1999-2000 and 2005-2006. Korea's aging rate is 1.5 times faster than Japan and 5 times faster than France.

This rapid aging creates social problems such as increased medical expenses, decreased role of seniors, alienation, or the like, and results in a rapid increase in patients with dementia.

According to the prevalence survey of the Ministry of Health and Welfare, the number of dementia patients in Korea is 540,000 in 2012, and is expected to increase rapidly to reach 840,000 in 2020. Korea's dementia population is the fastest growing in the world, and its social cost is projected to reach 1.5% of GDP in 2050.

Although several drugs may be used as a treatment for dementia, which is expected to increase rapidly, these drugs have the effect of slowing progression, not the underlying treatment of dementia. However, such drugs have a relatively high efficacy based on it being prescribed and used in the early stages of dementia.

Early prediction and early diagnosis of dementia may play a decisive role in alleviating dementia symptoms. Alleviating symptoms through the early prediction of dementia may reduce social and economic costs. In order to address the rapid increase of dementia patients and high social costs, early prediction of dementia disease is very urgent.

### SUMMARY

Accordingly, the present disclosure was devised. An object of the present disclosure is to provide an apparatus and method for processing data for predicting dementia through machine learning that may predict dementia early through machine learning and improve an early diagnosis rate of dementia for each individual.

According to the present disclosure, an apparatus for processing a user's medical data to be input into a machine learning device for predicting dementia is provided. The apparatus comprises a pre-processing unit for setting a value of each preset feature as a value to be input to the machine learning device based on the user's medical data and a data set configuration unit for generating a data set including the value of each feature set by the pre-processing unit. Each feature set in the pre-processing unit may comprise at least one group of features of a first group of features, a second group of features, a third group of features, and a fourth group of features. The first group of features may comprise at least one of hyperfunction of pituitary gland, hypofunction and other disorders of pituitary gland, other disorders of adrenal gland, and unspecified protein-energy malnutrition. The second group of features may comprise at least one of calculus of lower urinary tract, urethral stricture, other disorders of male genital organs, inflammatory disease of uterus, except cervix, and polyp of female genital tract. The third group of features may comprise at least one of kyphosis and lordosis, spinal osteochondrosis, and psoriatic and enteropathic arthropathies. The fourth group of features may comprise at least one of ascites, retention of urine, voice disturbances, malaise and fatigue, enlarged lymph nodes, and systemic inflammatory response syndrome.

According to the present disclosure, a method for processing a user's medical data to be input into a machine learning device for predicting dementia is provided. The method comprises pre-processing in which a value of each preset feature is set as a value to be input to the machine learning device based on the user's medical data and generating a data set including the value of each feature set by the pre-processing. Each feature set in the pre-processing unit may comprise at least one group of features of a first group of features, a second group of features, a third group of features, and a fourth group of features. The first group of features may comprise at least one of hyperfunction of pituitary gland, hypofunction and other disorders of pituitary gland, other disorders of adrenal gland, and unspecified protein-energy malnutrition. The second group of features may comprise at least one of calculus of lower urinary tract, urethral stricture, other disorders of male genital organs, inflammatory disease of uterus, except cervix, and polyp of female genital tract. The third group of features may comprise at least one of kyphosis and lordosis, spinal osteochondrosis, and psoriatic and enteropathic arthropathies. The fourth group of features may comprise at least one of ascites, retention of urine, voice disturbances, malaise and fatigue, enlarged lymph nodes, and systemic inflammatory response syndrome.

According to the present disclosure, a non-transitory recording medium readable by a computer system on which a program is recoded is provided. The program may be for executing a method for processing data for predicting dementia through machine learning. The method may comprise pre-processing in which a value of each preset feature is set as a value to be input to the machine learning device based on the user's medical data and generating a data set including the value of each feature set by the pre-processing.

Aspects of the presently disclosed technology are not restricted to those set forth herein. The above and other aspects of the presently disclosed technology will become more apparent to one of ordinary skill in the art to which the presently disclosed technology pertains by referencing the detailed description of the presently disclosed technology given below.

According to the present disclosure, optimal features are constructed using a user's medical data for each year among many factors that may be used for predicting dementia through learning, thereby enabling accurate dementia prediction and diagnosis.

Regarding to the user's medical data for each year, reliability may be further increased by using big data from an organization that collects and manages many individual health-related information, such as the Korea National Health Insurance Service (KNIS).

For example, since the experimental results show that the prediction results of observing a disease history of 7 years or less are the best, rather than observing medical information for a long period of time, the appropriate criteria are suggested for predicting dementia.

Precise dementia prediction may be prescribed in the early stages of dementia, so it may play a decisive role in alleviating dementia symptoms and social and economic costs may be reduced.

### BRIEF DESCRIPTION OF THE DRAWINGS

The above and other aspects and features of the presently disclosed technology will become more apparent by describing in detail exemplary embodiments thereof with reference to the attached drawings, in which:

FIG. 1 is an embodiment of an apparatus for processing data for predicting dementia according to the present disclosure;

FIG. 2 is an example for explaining the overall process of dementia prediction using machine learning according to the present disclosure;

FIG. 3 is an embodiment of a method for processing data for predicting dementia according to the present disclosure;

FIG. 4 is an example for explaining a process of dementia prediction process used in an experiment; and

FIG. 5 is an example for explaining a method for selecting an experimental object.

### DETAILED DESCRIPTION OF THE EMBODIMENTS

Hereinafter, embodiments of the present disclosure will be described with reference to the attached drawings. Advantages and features of the present disclosure and methods of accomplishing the same may be understood more readily by reference to the following detailed description of embodiments and the accompanying drawings. The present disclosure may, however, be embodied in many different forms and should not be construed as being limited to the embodiments set forth herein. Rather, these embodiments are provided so that this disclosure will be thorough and complete and will fully convey the technology of the disclosure to those skilled in the art, and the present disclosure will be defined by the appended claims.

In adding reference numerals to the components of each drawing, it should be noted that the same reference numerals are assigned to the same components as much as possible even though they are shown in different drawings. In addition, in describing the presently disclosed technology, based on it being determined that the detailed description of the related well-known configuration or function may obscure the gist of the presently disclosed technology, the detailed description thereof will be omitted.

Unless otherwise defined, all terms used in the present specification (including technical and scientific terms) may be used in a sense that can be commonly understood by those skilled in the art. In addition, the terms defined in the commonly used dictionaries are not ideally or excessively interpreted unless they are specifically defined clearly. The terminology used herein is for the purpose of describing embodiments and is not intended to be limiting of the presently disclosed technology. In this specification, the singular also includes the plural unless specifically stated otherwise in the phrase.

In addition, in describing the component of this presently disclosed technology, terms, such as first, second, A, B, (a), (b), can be used. These terms are for distinguishing the components from other components, and the nature or order of the components is not limited by the terms. If a component is described as being “connected,” “coupled” or “contacted” to another component, that component may be directly connected to or contacted with that other component, but it should be understood that another component also may be “connected,” “coupled” or “contacted” between each component.

Hereinafter, some embodiments of the presently disclosed technology will be described in detail with reference to the accompanying drawings.

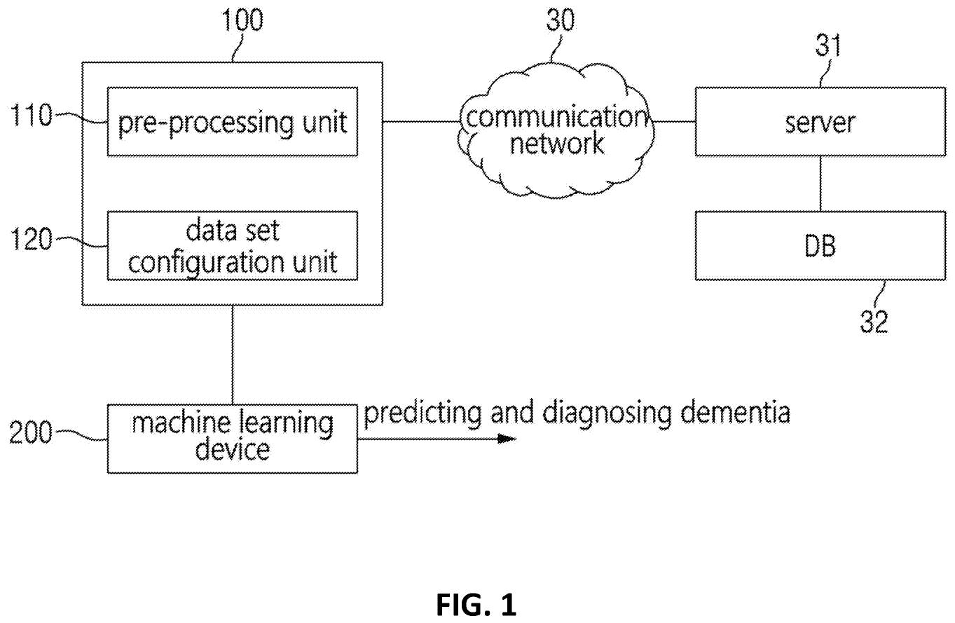

Referring to FIG. 1, an apparatus for processing data 100 for predicting dementia according to the present disclosure uses a data set including optimal features based on a user's medical data for each year (medical history) to predict dementia through machine learning.

For the machine learning, various tools may be used. Examples for the machine learning include, but are not limited to, the open source data mining program WEKA (Waikato environment for knowledge analysis) developed in the Java language.

The user's medical data for each year may include any information related to the user's health, in which in the present disclosure, it is configured to include at least information related to the user's disease (disease history).

A route of obtaining the user's medical data for each year may vary. As one example, the user's medical data for each year may be received from a server 31 managing the user's health information through a wide area communication network 30 such as an Internet network.

In Korea, an institution that may receive the user's medical data for each year may be the Korean National Health Insurance Service (KNHIS), in which the KNHIS operates a database 32 that manages big data by collecting all medical records in Korea under the national policy, and provides the information.

In addition, medical data for each user, which may be stored/maintained internally by an institution such as a hospital, may be used.

A pre-processing unit 110 sets a value of each preset feature as a value to be input to a machine learning device 200 based on the user's medical data for each year.

Here, pre-processing means that a value of each feature may be set to a value to be used for predicting dementia, and that data may be processed in a format used by the machine learning device 200 to be used for predicting dementia.

The pre-processing unit 110 performs pre-processing of at least the former. For example, assuming that a feature may be a hemoglobin value, the value of this feature item may be set to a value indicating normal or abnormal (e.g., ‘1’ or ‘0’) according to a reference value.

A data set configuration unit 120 configures a data set including the values of respective features set in the pre-processing unit 110. In other words, not all items belonging to the user's medical data for each year may be used for machine learning, but a combination of features determined as optimal may be used for the machine learning.

The data to be used for the machine learning may be a value for each year corresponding to each feature item determined as optimum, and may be a data set including these values. Data recorded in the user's medical data may be used as a value for each year. However, the value may be a value classified as the user's status or range for corresponding features, such as normal/abnormal, existence/non-existence, high/normal/low, upper/mid/low, or the like.

The optimal features may be those evaluated as optimal for predicting dementia using the machine learning. Which features may be most suitable for predicting dementia may be set in various ways. However, in examples of experiments related to the present disclosure, 80 features, which will be described in detail below, were determined as optimal. Here, the optimal feature may be configured to include at least information on the user's disease history.

With regard to the user's medical data for each year, it may be configured in various ways how to set a period of data to be input to the machine learning device 200. For example, it may be possible to set the user's medical data of the last 7 years or less as data to be input into the machine learning device 200.

In this embodiment, an object to be processed by the pre-processing unit 110 and the data set configuration unit 120 may be the user's medical data for the last 7 years or less. This may be according to the experimental results of the present disclosure, and observing for a long period of time unconditionally does not guarantee high predictive performance.

The most suitable features for predicting dementia may be variously configured, and it may be configured to include at least hyperfunction of pituitary gland, hypofunction and other disorders of pituitary gland, other disorders of adrenal gland, unspecified protein-energy malnutrition, calculus of lower urinary tract, urethral stricture, other disorders of male genital organs, inflammatory disease of uterus, except cervix, polyp of female genital tract, kyphosis and lordosis, spinal osteochondrosis, psoriatic and enteropathic arthropathies, ascites, retention of urine, voice disturbances, malaise and fatigue, enlarged lymph nodes, and systemic inflammatory response syndrome.

The features may be items evaluated as factors related to dementia newly discovered in the experiment of the present disclosure.

Further, in addition to the features, it may further include total cholesterol, hemoglobin, serum GOT, serum GPT, gamma GTP, other disorders of pancreatic internal secretion, vitamin D deficiency, other disorders of thyroid, malnutrition-related diabetes mellitus, dementia in Alzheimer disease, vascular dementia, mental and behavioural disorders due to use of alcohol, acute and transient psychotic disorders, unspecified nonorganic psychosis, unspecified dementia, bipolar affective disorder, depressive episode, delirium, not induced by alcohol and other psychoactive substances, eating disorders, psychological and behavioural factors associated with disorders or diseases classified elsewhere, other mental disorders due to brain damage and dysfunction and to physical disease, schizophrenia, Parkinson disease, secondary parkinsonism, parkinsonism in diseases classified elsewhere, Alzheimer disease, other degenerative diseases of nervous system NEC, epilepsy, status epilepticus, transient cerebral ischemic attacks and related syndromes, vascular syndromes of brain in cerebro-vascular diseases, disorders of other cranial nerves, hemiplegia, paraplegia and tetraplegia, other paralytic syndromes, hydrocephalus, other disorders of brain, other disorders of nervous system, NEC, other disorders of nervous system in diseases classified elsewhere, hypertensive renal disease, subsequent myocardial infarction, cerebral infarction, cerebrovascular disorders in diseases classified elsewhere, sequelae of cerebrovascular disease, aortic aneurysm and dissection, stroke, not specified as haemorrhage or infarction, acute nephritic syndrome, chronic kidney disease, glomerular disorders in diseases classified elsewhere, faecal incontinence, abnormalities of gait and mobility, unspecified urinary incontinence, somnolence, stupor and com, other symptoms and signs involving cognitive functions and awareness, other symptoms and signs involving general sensations and perceptions, symptoms and signs involving appearance and behavior, fracture of skull and facial bones, open wound of thorax, injury of other and unspecified intrathoracic organs, open wound of forearm, (5) fracture at wrist and hand level, fracture at wrist and hand level, and injury of muscle and tendon at hip and thigh level.

Tables 1 to 6 below show 80 optimal features.

TABLE 1

| No. | Classification | |
|---|---|---|
| 1 | GHE (general health | Total cholesterol |
| 2 | examinations) DB | Hemoglobin |
| 3 | | Serum GOT |
| 4 | | Serum GPT |
| 5 | | Gamma GTP |
| 6 | MT (medical | Other disorders of pancreatic |
| | treatments) DB | internal secretion |
| 7 | ICD code E: | Vitamin D deficiency |
| 8 | endocrine, | Other disorders of thyroid |
| 9 | nutritional, | Malnutrition-related diabetes mellitus |
| 10 | and metabolic | Hyperfunction of pituitary gland |
| 11 | diseases | Hypofunction and other disorders of |
| | | pituitary gland |
| 12 | | Other disorders of adrenal gland |
| 13 | | Unspecified protein-energy malnutrition |
| 14 | ICD code F: | Dementia in Alzheimer disease |
| 15 | mental and | Vascular dementia |
| 16 | behavior disorder | Mental and behavioural disorders due to |
| | | use of alcohol |
| 17 | | Acute and transient psychotic disorders |
| 18 | | Unspecified nonorganic psychosis |
| 19 | | Unspecified dementia |
| 20 | | Bipolar affective disorder |
| 21 | | Depressive episode |
| 22 | | Delirium, not induced by alcohol and other |
| | | psychoactive substances |
| 23 | | Eating disorders |
| 24 | | Psychological and behavioural factors |
| | | associated with disorders or diseases |
| | | classified elsewhere |
| 25 | | Other mental disorders due to brain |
| | | damage and dysfunction and to physical |
| | | disease |
| 26 | | Schizophrenia |
| 27 | ICD code G: | Parkinson disease |
| 28 | nervous | Secondary parkinsonism |
| 29 | system disease | Parkinsonism in diseases classified elsewhere |
| 30 | | Alzheimer disease |
| 31 | | Other degenerative diseases of nervous |
| | | system NEC |
| 32 | | Epilepsy |
| 33 | | Status epilepticus |
| 34 | | Transient cerebral ischemic attacks and |
| | | related syndromes |
| 35 | | Vascular syndromes of brain in |
| | | cerebro-vascular diseases |
| 36 | | Disorders of other cranial nerves |
| 37 | | Hemiplegia |
| 38 | | Paraplegia and tetraplegia |
| 39 | | Other paralytic syndromes |
| 40 | | Hydrocephalus |
| 41 | | Other disorders of brain |
| 42 | | Other disorders of nervous system, NEC |
| 43 | | Other disorders of nervous system in |
| | | diseases classified elsewhere |

TABLE 2

| | | |
|---|---|---|
| 44 | ICD code I: | Hypertensive renal disease |
| 45 | circulatory | Subsequent myocardial infarction |
| 46 | system diseases | Cerebral infarction |
| 47 | | Cerebrovascular disorders in diseases |
| | | classified elsewhere |
| 48 | | Sequelae of cerebrovascular disease |
| 49 | | Aortic aneurysm and dissection |
| 50 | | Stroke, not specified as haemorrhage or infarction |

TABLE 3

| | | |
|---|---|---|
| 51 | ICD code N: | Acute nephritic syndrome |
| 52 | urogenital | Chronic kidney disease |
| 53 | diseases | Glomerular disorders in diseases classified |
| | | elsewhere |
| 54 | | Calculus of lower urinary tract |
| 55 | | Urethral stricture |
| 56 | | Other disorders of male genital organs |
| 57 | | Inflammatory disease of uterus, except |
| | | cervix |
| 58 | | Polyp of female genital tract |

TABLE 4

| | | |
|---|---|---|
| 59 | ICD code M: diseases of the | Kyphosis and lordosis |
| 60 | musculoskeletal system and | Spinal osteochondrosis |
| 61 | connective tissue | Psoriatic and enteropathic |
| | | arthropathies |

TABLE 5

| | | |
|---|---|---|
| 62 | ICD code R: | Faecal incontinence |
| 63 | symptoms, | Abnormalities of gait and mobility |
| 64 | signs and | Unspecified urinary incontinence |
| 65 | abnormal | Somnolence, stupor and coma |
| 66 | clinical and | Other symptoms and signs involving |
| | laboratory | cognitive functions and awareness |
| 67 | findings, | Other symptoms and signs involving |
| | NEC | general sensations and perceptions |
| 68 | | Symptoms and signs involving appearance |
| | | and behavior |
| 69 | | Ascites |
| 70 | | Retention of urine |
| 71 | | Voice disturbances |
| 72 | | Malaise and fatigue |
| 73 | | Enlarged lymph nodes |
| 74 | | Systemic Inflammatory Response Syndrome |

TABLE 6

| | | |
|---|---|---|
| 75 | ICD code S: | Fracture of skull and facial bones |
| 76 | other | Open wound of thorax |
| 77 | consequences | Injury of other and unspecified |
| | of injury, | intrathoracic organs |
| 78 | addiction, | Open wound of forearm, (5) Fracture at |
| | and other | wrist and hand level |
| 79 | external | Fracture at wrist and hand level |
| 80 | causes | Injury of muscle and tendon at hip and |
| | | thigh level |

Here, the pre-processing unit 110 may be configured such that total cholesterol among the features may be set to a value indicating that it may be normal e.g., 40 to 229 mg/dL, and may be set to a value indicating that it is abnormal e.g., 230 to 999 mg/dL.

It may be configured such that, in the case of men, hemoglobin may be set to a value indicating that it is normal e.g., 12 to 16.5 g/dL, and may be set to a value indicating that it is abnormal e.g., 0 g/dL or more and less than 12 g/dL. It may be configured such that, in the case of women, hemoglobin may be set to a value indicating that it is normal e.g., 10 to 15.5 g/dL, and may be set to a value indicating that it is abnormal e.g., 0 g/dL or more and less than 10 g/dL.

It may be configured such that, in the case of men, gamma GPT may be set to a value indicating that it is normal e.g., 11 to 77 U/L, and may be set to a value indicating that it is abnormal e.g., 78 to 999 U/L.

It may be configured such that, in the case of women, gamma GPT may be set to a value indicating that it is normal e.g., 8 to 45 U/L, and may be set to a value indicating that it is abnormal e.g., 46 to 999 U/L.

Also, the pre-processing unit 110 may set features other than total cholesterol, hemoglobin, serum GOT, serum GPT, and gamma GPT among the features to a value indicating one of the presence and absence of that disease.

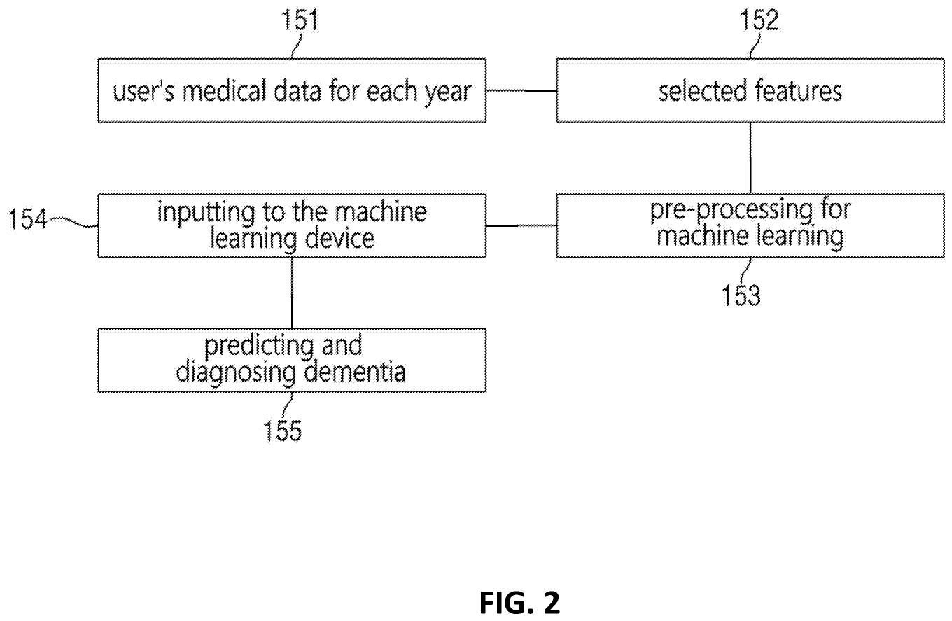

FIG. 2 is an example for explaining the overall overview of dementia prediction using machine learning according to the present disclosure, in which input user's medical data for each year 151 may not all be used, but 80 selected features shown in Tables 1 to 6 may be used (152). The values for the selected features may be set to values suitable for the machine learning through pre-processing (153), and then input to the machine learning device (154), and may be processed according to an appropriate algorithm to predict or diagnose dementia (155).

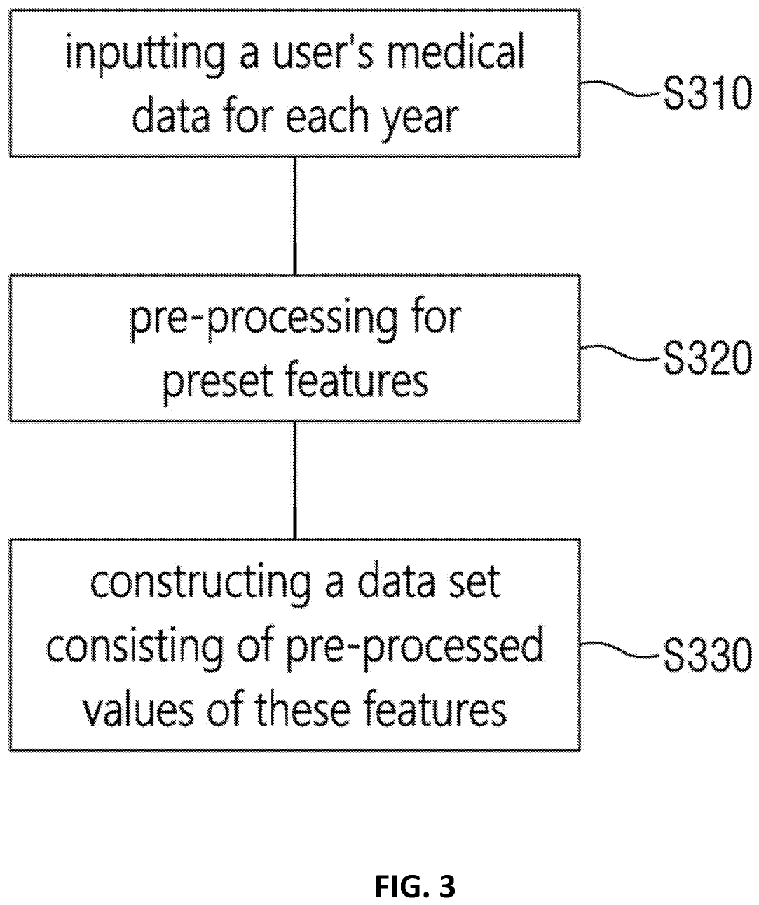

Referring to FIG. 3, an embodiment of a method for processing data for predicting dementia according to the present disclosure will be described.

First, a user's medical data for each year to be processed may be input (S310). The user's medical data for each year may include any information related to the user's health, in which in the present disclosure, it may be configured to include at least information related to the user's disease (disease history).

A route for receiving the user's medical data for each year may vary. As one example, the user's medical data for each year may be received in real time from a server managing user health information through a wide area communication network such as an Internet network, and may be input from a file received or obtained in advance and saved. In addition, it may be possible to receive the user's medical data for each year which may be stored/managed by an institution itself such as a hospital through an internal communication network.

Now, based on the user's medical data for each year, a value of each preset feature may be set as a value to be input to a machine learning device (S320).

Step S320 may be a process of setting a value of each preset feature as a value to be actually input to the machine learning device based on the user's medical data for each year input through step S310. As an example, the values of each feature may be set to a value in a range determined for predicting dementia.

The features for setting the value in step S320 refer to features evaluated as optimal for predicting dementia using the machine learning.

Which features may be most suitable for predicting dementia may be set in various ways. However, optimal features in the present disclosure may be configured to include at least information on the user's disease history. The most examples of the optimal features may be the 80 features shown in Tables 1 to 6 above.

A data set including the values of the respective features processed in step S320 may be constructed (S330).

In other words, not all items belonging to the user's medical data for each year may be used for machine learning, but a combination of features determined as optimal may be used for the machine learning. The data to be used for the machine learning may be a value for each year corresponding to each feature item determined as optimum, and may be a data set including these values.

Here, a value recorded in the user's medical data may be used as a value for each year. However, the value may be a value classified according to the user's status or range for corresponding features, such as normal/abnormal, existence/non-existence, high/normal/low, upper/mid/low, or the like.

With regard to the user's medical data for each year, it may be configured in various ways how to set a period of data to be input to the machine learning device. In this regard, it may be possible to set the user's medical data of the last 7 years or less as data to be input into the machine learning device.

In this embodiment, an object to be processed in steps S320 and S330 may be the user's medical data for the last 7 years or less.

In step S320, total cholesterol, hemoglobin, serum GOT, serum GPT, gamma GPT, or the like among the features may be classified as normal/abnormal, and other features may be set to a value indicating the presence or absence of that disease.

Here, in step S320, it may be configured such that total cholesterol among the features may be set to a value indicating that it is normal e.g., 40 to 229 mg/dL, and may be set to a value indicating that it is abnormal e.g., 230 to 999 mg/dL.

It may be configured such that, in the case of men, hemoglobin may be set to a value indicating that it is normal e.g., 12 to 16.5 g/dL, and may be set to a value indicating that it is abnormal e.g., 0 g/dL or more and less than 12 g/dL. It may be configured such that, in the case of women, hemoglobin may be set to a value indicating that it is normal e.g., 10 to 15.5 g/dL, and may be set to a value indicating that it is abnormal e.g., 0 g/dL or more and less than 10 g/dL.

It may be configured such that, in the case of men, gamma GPT may be set to a value indicating that it is normal e.g., 11 to 77 U/L, and may be set to a value indicating that it is abnormal e.g., 78 to 999 U/L.

It may be configured such that, in the case of women, gamma GPT may be set to a value indicating that it is normal e.g., 8 to 45 U/L, and may be set to a value indicating that it is abnormal e.g., 46 to 999 U/L.

A method for processing data for predicting dementia through machine learning according to the present disclosure may be embodied as computer readable codes on a computer readable recording medium.

The computer readable recording medium includes all types of recording devices in which data readable by a computer system may be stored.

For example, it may include a ROM, RAM, CD-ROM, magnetic tape, floppy disk, or optical data storage device. In addition, the computer readable recording medium may be distributed over network coupled computer systems so that the computer readable code may be stored and executed in a distributed fashion.

Concrete Experiment

Now, experimental examples related to the present disclosure will be described.

### 1. In this Experiment, Dementia was Predicted Using Data from the KNHIS (Korea National Health Insurance Service), which May be Representative of the Entire Population of Korean. Since the KNHIS Automatically Collects all Medical Records in Korea Under National Policy, a Database of the KNHIS May Represent the Entire Population of Korea

Among them, a senior cohort database includes information such as insurance eligibility, income, records of medical services benefits, medical records, detail of long-term care and health checks, or the like. A PIE (participant's insurance eligibility) database includes demographic information, socio-economic levels and other information, or the like. An MT (medical treatments) database includes information on medical subjects and medical illnesses, or the like, and a GHE (general health examinations) database includes detail of health checks from anthropometric measurements to past history. An MCI (medical care institution) database includes information such as the type, region, and founded time of medical use nursing care institutions, or the like, and information such as the number of hospital beds, the number of doctors, equipment holding status in a nursing care institution, or the like. Finally, an LCI (long-term care insurance) database includes long-term care application and decision results, a doctor's opinions, such as a certified apprentice, information on long-term care facilities, or the like.

The Korea national health insurance service senior cohort (KNHIS-SC) database provides a variety of variables for highly reliable data composition and samples.

### 2. In this Experiment, it was Intended to Derive the Most Appropriate Features and Observation Periods for Predicting Dementia Through Demographics, Health Checks, and Personalized History Information in the KNHIS-SC Database

A. Workflow

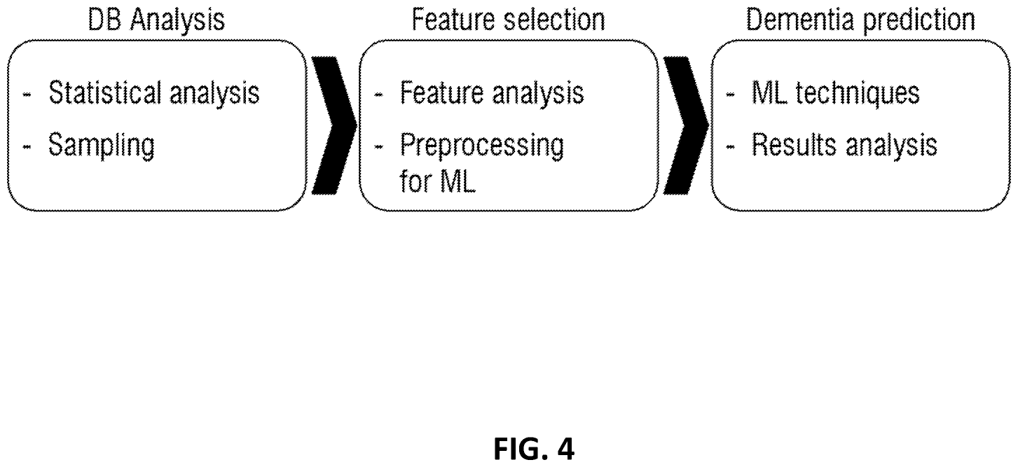

For the prediction of dementia, sociodemographic, detail of health checks, and medical records belonging to a personal medical history were used. FIG. 4 shows a process for predicting dementia, in which the KNHIS-SC database was analyzed to extract samples for experiments, select features, and perform pre-processing to apply them to a machine learning technique. By applying the machine learning technique, a combination of optimal features may be derived, and an optimal prediction model may be built.

B. Feature Selection

i) Feature Analysis

The personal medical history, which may be widely used to predict dementia, includes social demographic data, lifestyle, personal disease history, biophysical characteristics, or the like. In this experiment, these items were selected as features for application to the machine learning technique.

Specifically, among items in the KNHIS-SC database, social demographic data of the PIE-DB (e.g., sex, age, income quintile), anthropometric data of the GHE-DB (e.g., height, weight, body mass index, waist, blood pressure highest, blood pressure lowest), blood test results (e.g., blood glucose level before meals and levels of total cholesterol, hemoglobin, serum GOT, serum GPT, and gamma-GTP), urine test results, a personal past disease history (e.g., stroke, heart disease, high blood pressure, diabetes, hyperlipidemia, phthisis, cancer), a family past disease history (e.g., stroke, heart disease, high blood pressure, diabetes, cancer), smoking or nonsmoking, and a disease history in the MT-DB, or the like were selected.

‘on medical diseases’ of the MT-DB consists of about 2,600 3-digit international classification of disease (ICD) codes, in which the ICD code consists of 26 alphabet chapters in the first digit and two digits from 00 to 99 (extended disease group). This ICD code was selected as a feature item.

ii) Pre-Processing for Machine Learning

In the pre-processing, items selected from the PIE-DB, the MT-DB, and the GHE-DB were processed into a feature form suitable for machine learning.

For example, in the PIE-DB, a sex may be classified into a male and a female. An age was divided into 7 levels, and income was divided into 3 levels. In the MT-DB, based on the ICD code, data depending on the presence or absence of diseases and the change in a time series pattern of the diseases were used. Finally, in the GHE-DB, a height was divided into 13 levels with 101 cm to 230 cm in 10 cm increments, and a body weight was divided into 11 levels with 26 kg to 300 kg in 5 kg increments.

Variables for knowing the abnormality of a body through the results of a waist, body mass index, blood test, urine test, or the like were divided into normal and abnormal ranges according to the health examination practice criteria (Ministry of Health and Welfare Notification No. 2016-11).

Table 7 below shows normal/abnormal range criteria of an item in the GHE-DB.

TABLE 7

| No. | Feature | Status | |
|---|---|---|---|
| No. | Feature | Normal | Abnormal |
| 1 | Body Mass Index(kg/) | 0~29 | 30~300 |
| 2 | WAIST(cm) | male: 50~90, | male: 90~130, |
| | | female: 50~85 | female: 85~130 |
| 3 | blood pressure | 60~139 | 140~400 |
| | highest (mmhg) | | |
| 4 | blood pressure | 40~89 | 90~250 |
| | lowest (mmhg) | | |
| 5 | Blood sugar before | 25~125 | 126~999 |
| | meals(g/dL) | | |
| 6 | Total cholesterol(mg/dL) | 40~229 | 230~999 |
| 7 | hemoglobin(g/dL) | male: 12~16.5, | male: 0~12, |
| | | female: 10~15.5 | female: 0~10 |
| 8 | Urine protein | Negative | Positive |
| 9 | Serum GOT(U/L) | 0~50 | 51~999 |
| 10 | Serum GPT(U/L) | 0~45 | 46~999 |
| 11 | Gamma GTP(U/L) | Male: 11~77, | Male: 78~999, |
| | | female: 8~45 | female: 46~999 |

Each feature was created for each year from 2003 to 2013 to identify time series patterns. For example, for the features of the GHE-DB, the increase and decrease of the change and the direction of normal/abnormal change were featured for each year and each feature item to measure the change compared to 2013 for each year.

Each feature may be organized by sample, and was divided into dementia (DM) and NC (normal control) with or without F00 (dementia in Alzheimer disease; G30), F01 (Vascular dementia), F02 (dementia in other diseases classified elsewhere (dementia with Lewy bodies, Creutzfeldt-Jakob disease, and dementia in human immunodeficiency virus [HIV] disease may be included)), F03 (unspecified dementia), and G30, which may be ICD codes as of 2013. Here, criteria of ‘F00, F01, F02, F03, G30’ may be dementia diagnostic codes used in Korea to provide medical subsidies to dementia patients.

C. Approach (Longitudinal Study-Based Dementia Prediction)

In this experiment, it was intended to prove the following two hypotheses. First, a personal medical history will have an influence on improving dementia prediction performance. Second, a personal disease history will be the relevant information among medical histories. The personal medical history from 2003 to 2013 was used to prove the first hypothesis. In order to compare the performance between items of the medical histories, a set of experiments consisting of information from 2013 was set as the baseline.

In this experiment, an experiment was also conducted to determine the best observation period for predicting dementia. It consists of a set of experiments of the last 3 years, 5 years, 7 years, 9 years, and 11 years, including 2013, and the changes in the experimental results may be compared. In addition to comparing the simple increase or decrease of the items, the comparison was made considering the change in the state of the normal/abnormal range.

In order to identify the second hypothesis, which may be the impact of the personal disease history, the best combination of features was constructed through comparison with other features.

### 3. Experiment

A. Sampling

The following rules were applied to apply the machine learning technique.

(i) For people over 65, the KNHIS provides free health checks once every two years. Samples were taken every two years between 2003 and 2013 for the use of health check results. (ii) 11,443 people were obtained as a result of application of (i), in which it consists of 850 DM (dementia patients) and 10,593 NC (normal control)(control group). (iii) 850 NC and 850 DM were randomly extracted and used in the experiment.

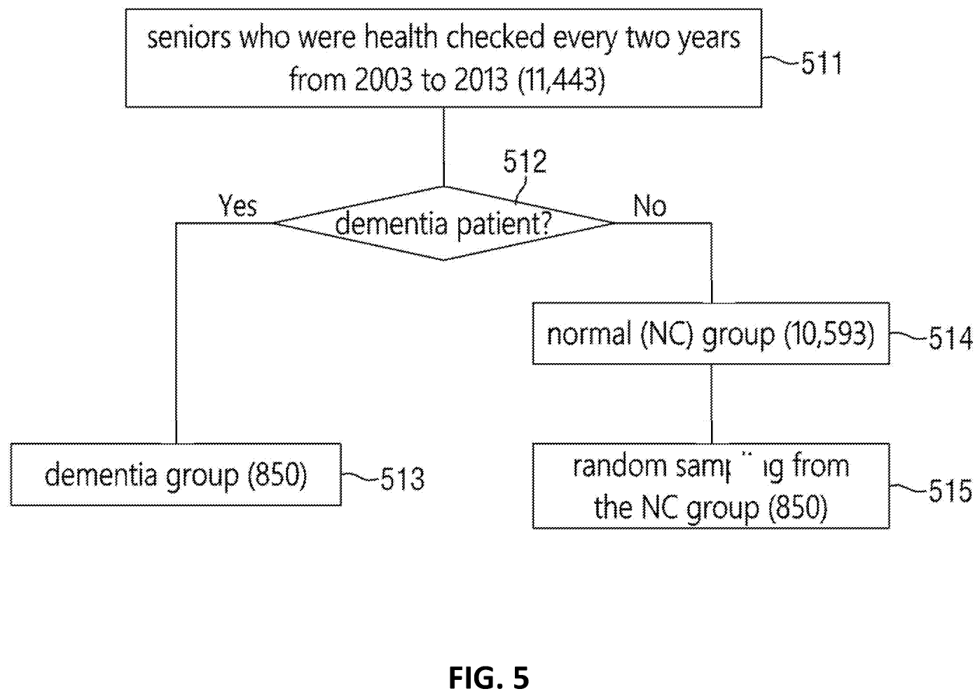

Referring to FIG. 5, a study sample was extracted for seniors who had a medical examination in 2013. In 2013, 82,613 people had health checks, of which 11,443 were health checked every other year from 2003 to 2013 (511). 850 patients with dementia (DM), which may be samples having F00, F01, F02, F03, and G30 codes among the ICD codes, were extracted (512 and 513). Among seniors who were not dementia (512), NC was 10,593 (514), among which 850 experimental samples were constructed by random sampling (515).

B. Experiment Setting

In order to explore appropriate machine learning techniques for predicting dementia and deriving a model for predicting dementia, as described above, 850 elderly people with dementia and 850 elderly people without dementia were selected from the KNHIS-SC database, and 4 types in the PIE-DB, 70 types in the GHE-DB, and 2600 in the MT-DB were selected as the features.

Table 8 shows the features of the baseline, and the features of 2013 were used in a basic experiment to prove the validity of time series information.

TABLE 8

| Year | DB | Features |
|---|---|---|
| 2013 | PIE-DB | Sex, age, income quintile |
| 2013 | GHE-DB | height, weight, body mass index, waist, blood |
| | | pressure highest, blood pressure lowest, Blood |
| | | sugar before meals, Total cholesterol, hemoglobin, |
| | | Urine protein, Serum GOT, Serum GPT, Gamma GTP, |
| | | History of personal illness: stroke, heart |
| | | disease, high blood pressure, diabetes, |
| | | hyperlipidemia, phthisis, cancer), History of |
| | | family illness: stroke, heart disease, high blood |
| | | pressure, diabetes, cancer |
| 2013 | MT-DB | ICD-code |

An experiment set for each year to check the time series information was constructed by adding the features of each year to the baseline as shown in Table 9.

TABLE 9

| DB | Features (Baseline + longitudinal data) | |
|---|---|---|
| PIE-DB | Features of | increase/decrease compared to 2013 |
| | Baseline | [income quintile] |
| | | status changing compared to 2013 |
| | | [income quintile] |
| GHE-DB | Features of | increase/decrease compared to 2013 |
| | Baseline | [height/weight/body mass index/waist/ |
| | | blood pressure highest/blood pressure |
| | | lowest/Blood sugar before meals/Total |
| | | cholesterol/hemoglobin/Urine protein/ |
| | | Serum GOT/Serum GPT/Gamma GTP/History |
| | | of personal illness: stroke, heart disease, |
| | | high blood pressure, diabetes, |
| | | hyperlipidemia, phthisis, cancer/History |
| | | of family illness: stroke, heart disease, |
| | | high blood pressure, diabetes, cancer] |
| | | status changing compared to 2013 |
| | | [body mass index/waist/blood pressure |
| | | highest/blood pressure lowest/Blood sugar |
| | | before meals/Total cholesterol/ |
| | | hemoglobin/Urine protein/Serum GOT/ |
| | | Serum GPT/Gamma GTP/History of personal |
| | | illness: stroke, heart disease, high |
| | | blood pressure, diabetes, hyperlipidemia, |
| | | phthisis, cancer/History of family |
| | | illness: stroke, heart disease, high |
| | | blood pressure, diabetes, cancer] |
| MT-DB | Features of | Yearly Information on medical diseases |
| | Baseline | diagnosis |

Five experimental sets from 2003 to 2011 in two-year increments were made from 2013 to the last 3, 5, 7, 9, and 11 years, in which each set were consisted of measuring the degree of change by year based on whether the increase/decrease compared to 2013 or the normal/abnormal state change compared to 2013, and whether the ICD code may be diagnosed by year, depending on the nature of the features.

Two experiments were conducted in the tested approach to build a dementia prediction model that focuses on a personal medical history, and to determine the best way to use an optimal personal medical history period.

First, a longitudinal model 1 used a primary disease group for the personal disease history, and a longitudinal model 2 used an extended disease group. Through the above experiment, it was decided that the best way would be to use the personal disease history. Further, in order to prove the effectiveness of the personal medical history, a basic experiment was established using a function for one year (2013). In addition, in the experiment, periods were compared to determine the optimal period of the personal medical history for predicting dementia.

C. Methodology/Verification/Measurement

This experiment may not be focused on algorithm analysis of the machine learning, but it focuses on which feature combinations make a good prediction model. The WEKA, which may easily compare and analyze the influence on the features through existing algorithms, was used.

The WEKA includes most of the known algorithms, and has most of the functions for data mining, from feature selection to model evaluation. It may be useful for academic purposes.

First, gain ration attribute evaluation was used for the feature selection. An SVM (support vector machine), which may be one of the machine learning methods provided by the WEKA, was used.

Weka.classifiers.functions.SMO was used as an algorithm, Logistic was used as a calibrator, and RBFKernel (C=1.0, E=1.0) was used as a kernel. Using the K-fold cross-validation method, the model was verified with a 10-fold cross-validation method.

Evaluation measures were performed using Precision, Recall, and F-measure.

### 4. Result

Longitudinal features reflected time-series changes from 2002-2012 in baseline features.

Table 10 shows the results of the longitudinal model 1 and the baseline model. For the longitudinal model 1, the ‘primary disease group’ of the PT-DB, the GHE-DB, and the MT-DB was used. The baseline result was 69.0% F-measure, and the longitudinal model showed that the F-measure increased by about 1.3% p to 4.1% p. The 2009-2013 model showed the highest predictive power with 73.1% F-measure.

TABLE 10

| | Baseline | Longitudinal model 1 | | | | |
|---|---|---|---|---|---|---|
| Year | 2013 | 2003-2013 | 2005-2013 | 2007-2013 | 2009-2013 | 2011-2013 |
| #Feature | 55 | 366 | 314 | 262 | 210 | 158 |
| True positive | 614 | 638 | 613 | 630 | 648 | 648 |
| False positive | 317 | 285 | 280 | 282 | 274 | 292 |
| True negative | 533 | 565 | 570 | 568 | 576 | 558 |
| False negative | 236 | 212 | 237 | 220 | 202 | 202 |
| Accuracy (%) | 67.5 | 70.8 | 69.6 | 70.5 | 72.0 | 70.9 |
| Precision (%) | 66.0 | 69.1 | 68.6 | 69.1 | 70.3 | 68.9 |
| Recall (%) | 72.2 | 75.1 | 72.1 | 74.1 | 76.2 | 76.2 |
| F-measure (%) | 69.0 | 72.0 | 70.3 | 71.5 | 73.1 | 72.4 |

In the longitudinal model 2, instead of the primary disease group, the extended disease group (primary disease group E extended to E00, E01, E98, E99; each a primary disease group extended to 100 codes of extended disease group) was used. In the longitudinal model 2, the model was searched based on a yearly model, and Table 11 shows a result of the longitudinal model 2 compared to the baseline.

TABLE 11

| | Baseline | Longitudinal model 2 | | | | |
|---|---|---|---|---|---|---|
| Year | 2013 | 2003-2013 | 2005-2013 | 2007-2013 | 2009-2013 | 2011-2013 |
| #Feature | 55 | 8,106 | 6,506 | 4,906 | 3,306 | 1,706 |
| True positive | 614 | 611 | 606 | 609 | 621 | 614 |
| False positive | 317 | 188 | 160 | 135 | 134 | 110 |
| True negative | 533 | 662 | 690 | 715 | 716 | 740 |
| False negative | 236 | 239 | 244 | 241 | 229 | 236 |
| Accuracy (%) | 67.5 | 74.9 | 76.2 | 77.9 | 78.6 | 79.6 |
| Precision (%) | 66.0 | 76.5 | 79.1 | 81.9 | 82.3 | 84.8 |
| Recall (%) | 72.2 | 71.9 | 71.3 | 71.6 | 73.1 | 72.2 |
| F-measure (%) | 69.0 | 74.1 | 75.0 | 76.4 | 77.4 | 78.0 |

In addition, for optimization, the relative influence of the features was extracted using a gain ratio attribute evaluation method, and characteristics with high influence were sequentially collected. After that, a combination of all the features was tired and the best combination was found.

A longitudinal model 3 uses the best combination of the features shown in Tables 1 to 6. The results may be shown in Table 12, and a 2007-2013 model showed the best performance with the F-measure of 80.9%.

TABLE 12

| | Baseline | Longitudinal model 3 | | | | |
|---|---|---|---|---|---|---|
| Year | 2013 | 2003-2013 | 2005-2013 | 2007-2013 | 2009-2013 | 2011-2013 |
| #Feature | 55 | 709 | 559 | 409 | 259 | 113 |
| True positive | 614 | 623 | 625 | 633 | 619 | 611 |
| False positive | 317 | 78 | 79 | 82 | 69 | 67 |
| True negative | 533 | 772 | 771 | 768 | 781 | 783 |
| False negative | 236 | 227 | 225 | 217 | 231 | 239 |
| Accuracy (%) | 67.5 | 82.1 | 82.1 | 82.4 | 82.4 | 82.0 |
| Precision (%) | 66.0 | 88.9 | 88.8 | 88.5 | 90.0 | 90.1 |
| Recall (%) | 72.2 | 73.3 | 73.5 | 74.5 | 72.8 | 71.9 |
| F-measure (%) | 69.0 | 80.3 | 80.4 | 80.9 | 80.5 | 80.0 |

Tables 1 to 6 may be the best combination of the features obtained from the longitudinal model 3, in which it includes 5 attributes associated with blood tests of the GHE-DB and 75 characteristics of the extended disease group associated with the MT-DB. The features of the primary disease groups F and G include dementia-related diseases known through prior studies, and the features of the basic disease group M include dementia-related diseases newly detected through this experiment. In addition, the features of the basic disease groups S and I indicate anesthesia surgery and circulatory system diseases. In addition to disease characteristics, GHE function also influenced the prediction of dementia.

Blood test results of total cholesterol, hemoglobin, serum GOT, serum GPT, and gamma GTP may be the features for predicting dementia of the GHE-DB.

### 5. Conclusion

Using the KNHIS-SC database and the machine learning technique, a dementia prediction model for all Koreans was derived. Various features were analyzed and optimized to improve dementia prediction performance. Several experiments have shown that the personal disease history has a promising performance in predicting dementia. This experiment was the first attempt to build a dementia prediction model based on the entire population sample of Koreans, and may be relevant because it has demonstrated very good performance (80.9% F-measure).

The results of this experiment showed that the personal medical history may be used to predict dementia, and showed that the 7-year period and the 3-year period may be optimal observation periods. Relatively recent medical information was more effective in predicting dementia. In other words, it shows that longer observation periods may not improve performance. In addition, 18 new diseases that may be related to dementia were detected.

This experiment focuses on improving the performance of individual dementia diagnosis. However, experimental results may contribute to reducing the incidence of dementia, not only in Koreans, but also globally.

In the above description, it may be described that all the components constituting the embodiments of the present disclosure may be combined or operated as one, but the technical features of the present disclosure may be not limited to these embodiments. That is, within the scope of the present disclosure, all of the components may be selectively combined and operated in one or more combinations.

Although the operations may be shown in an order in the drawings, those skilled in the art will appreciate that many variations and modifications can be made to the embodiments without substantially departing from the principles of the presently disclosed technology. The disclosed embodiments of the presently disclosed technology may be used in a generic and descriptive sense and not for purposes of limitation. The scope of protection of the presently disclosed technology should be interpreted by the following claims, and all technical ideas within the scope equivalent thereto should be construed as being included in the scope of the technical idea defined by the present disclosure.

What is claimed is:

1. An apparatus for processing a user's medical data to be input into a machine learning device to predict dementia, comprising:
a pre-processing unit for setting a value of each preset feature as a value to be input to the machine learning device based on the user's medical data; and
a data set configuration unit for generating a data set including the value of each feature set by the pre-processing unit,
wherein each feature set in the pre-processing unit comprises at least one group of features of a first group of features, a second group of features, a third group of features, and a fourth group of features,
wherein the first group of features comprises at least one of hyperfunction of a pituitary gland, hypofunction and other disorders of the pituitary gland, other disorders of adrenal gland, and unspecified protein-energy malnutrition,
wherein the second group of features comprises at least one of calculus of lower urinary tract, urethral stricture, other disorders of male genital organs, inflammatory disease of uterus, except cervix and polyp of female genital tract,
wherein the third group of features comprises at least one of kyphosis and lordosis, spinal osteochondrosis, and psoriatic and enteropathic arthropathies, and
wherein the fourth group of features comprises at least one of ascites, retention of urine, voice disturbances, malaise and fatigue, enlarged lymph nodes, and systemic inflammatory response syndrome.

2. The apparatus of claim 1, wherein the user's medical data is received from a server managing the user's medical data through a communication network.

3. The apparatus of claim 1, wherein the data set constituted by the data set configuration unit comprises the user's medical information for each year in the last 7 years or less.

4. The apparatus of claim 1, wherein the preset feature further comprises hyperfunction of pituitary gland, hypofunction and other disorders of pituitary gland, other disorders of adrenal gland, unspecified protein-energy malnutrition, calculus of lower urinary tract, urethral stricture, other disorders of male genital organs, inflammatory disease of uterus, except cervix, polyp of female genital tract, kyphosis and lordosis, spinal osteochondrosis, psoriatic and enteropathic arthropathies, ascites, retention of urine, voice disturbances, malaise and fatigue, enlarged lymph nodes, and systemic inflammatory response syndrome.

5. The apparatus of claim 1, wherein the preset feature further comprises total cholesterol, hemoglobin, serum GOT, serum GPT, gamma GTP, other disorders of pancreatic internal secretion, vitamin D deficiency, other disorders of thyroid, malnutrition-related diabetes mellitus, dementia in Alzheimer disease, vascular dementia, mental and behavioural disorders due to use of alcohol, acute and transient psychotic disorders, unspecified nonorganic psychosis, unspecified dementia, bipolar affective disorder, depressive episode, delirium, not induced by alcohol and other psychoactive substances, eating disorders, psychological and behavioural factors associated with disorders or diseases classified elsewhere, other mental disorders due to brain damage and dysfunction and to physical disease, schizophrenia, Parkinson disease, secondary parkinsonism, parkinsonism in diseases classified elsewhere, Alzheimer disease, other degenerative diseases of nervous system NEC, epilepsy, status epilepticus, transient cerebral ischemic attacks and related syndromes, vascular syndromes of brain in cerebro-vascular diseases, disorders of other cranial nerves, hemiplegia, paraplegia and tetraplegia, other paralytic syndromes, hydrocephalus, other disorders of brain, other disorders of nervous system, NEC, other disorders of nervous system in diseases classified elsewhere, hypertensive renal disease, subsequent myocardial infarction, cerebral infarction, cerebrovascular disorders in diseases classified elsewhere, sequelae of cerebrovascular disease, aortic aneurysm and dissection, stroke, not specified as haemorrhage or infarction, acute nephritic syndrome, chronic kidney disease, glomerular disorders in diseases classified elsewhere, faecal incontinence, abnormalities of gait and mobility, unspecified urinary incontinence, somnolence, stupor and coma, other symptoms and signs involving cognitive functions and awareness, other symptoms and signs involving general sensations and perceptions, symptoms and signs involving appearance and behavior, fracture of skull and facial bones, open wound of thorax, injury of other and unspecified intrathoracic organs, open wound of forearm, (5) fracture at wrist and hand level, fracture at wrist and hand level, and injury of muscle and tendon at hip and thigh level.

6. The apparatus of claim 5, wherein the pre-processing unit sets such that, among the features,
total cholesterol is set to a value indicating that it is normal between 40 and 229 mg/dL, and is set to a value indicating that it is abnormal between 230 and 999 mg/dL,
hemoglobin is set to a value indicating that it is normal between 12 and 16.5 g/dL, and is set to a value indicating that it is abnormal between 0 g/dL and 12 g/dL, in the case of men, and is set to a value indicating that it is normal between 10 and 15.5 g/dL, and is set to a value indicating that it is abnormal between 0 g/dL and 10 g/dL, in the case of women,
serum GOT is set to a value indicating that it is normal between 0 and 50 U/L, and is set to a value indicating that it is abnormal between 51 and 999 U/L,
serum GPT is set to a value indicating that it is normal between 0 and 45 U/L, and is set to a value indicating that it is abnormal between 46 and 999 U/L, and
gamma GPT is set to a value indicating that it is normal between 11 and 77 U/L, and is set to a value indicating that it is abnormal between 78 and 999 U/L, in the case of men, and is set to a value indicating that it is normal between 8 and 45 U/L, and is set to a value indicating that it is abnormal between 46 and 999 U/L, in the case of women.

7. The apparatus of claim 6, wherein the pre-processing unit sets features other than the total cholesterol, the hemoglobin, the serum GOT, the serum GPT, and the gamma GPT among the features to a value indicating one of the presence and absence of that disease.

8. A method for processing a user's medical data to be input into a machine learning device to predict dementia, comprising:
pre-processing in which a value of each preset feature is set as a value to be input to the machine learning device based on the user's medical data; and
generating a data set including the value of each feature set by the pre-processing,
wherein each feature set in the pre-processing unit comprises at least one group of features of a first group of features, a second group of features, a third group of features, and a fourth group of features,
wherein the first group of features comprises at least one of hyperfunction of a pituitary gland, hypofunction and other disorders of the pituitary gland, other disorders of adrenal gland, and unspecified protein-energy malnutrition,
wherein the second group of features comprises at least one of calculus of lower urinary tract, urethral stricture, other disorders of male genital organs, inflammatory disease of uterus, except cervix, and polyp of female genital tract,
wherein the third group of features comprises at least one of kyphosis and lordosis, spinal osteochondrosis, and psoriatic and enteropathic arthropathies, and
wherein the fourth group of features comprises at least one of ascites, retention of urine, voice disturbances, malaise and fatigue, enlarged lymph nodes, and systemic inflammatory response syndrome.

9. The method of claim 8, wherein the user's medical data is received from a server managing the user's medical data through a communication network.

10. The method of claim 8, wherein the data set comprises the user's medical information for each year in the last 7 years or less.

11. The method of claim 8, wherein the preset feature further comprises hyperfunction of pituitary gland, hypofunction and other disorders of pituitary gland, other disorders of adrenal gland, unspecified protein-energy malnutrition, calculus of lower urinary tract, urethral stricture, other disorders of male genital organs, inflammatory disease of uterus, except cervix, polyp of female genital tract, kyphosis and lordosis, spinal osteochondrosis, psoriatic and enteropathic arthropathies, ascites, retention of urine, voice disturbances, malaise and fatigue, enlarged lymph nodes, and systemic inflammatory response syndrome.

12. The method of claim 8, wherein the preset feature further comprises total cholesterol, hemoglobin, serum GOT, serum GPT, gamma GTP, other disorders of pancreatic internal secretion, vitamin D deficiency, other disorders of thyroid, malnutrition-related diabetes mellitus, dementia in Alzheimer disease, vascular dementia, mental and behavioural disorders due to use of alcohol, acute and transient psychotic disorders, unspecified nonorganic psychosis, unspecified dementia, bipolar affective disorder, depressive episode, delirium, not induced by alcohol and other psychoactive substances, eating disorders, psychological and behavioural factors associated with disorders or diseases classified elsewhere, other mental disorders due to brain damage and dysfunction and to physical disease, schizophrenia, Parkinson disease, secondary parkinsonism, parkinsonism in diseases classified elsewhere, Alzheimer disease, other degenerative diseases of nervous system NEC, epilepsy, status epilepticus, transient cerebral ischemic attacks and related syndromes, vascular syndromes of brain in cerebro-vascular diseases, disorders of other cranial nerves, hemiplegia, paraplegia and tetraplegia, other paralytic syndromes, hydrocephalus, other disorders of brain, other disorders of nervous system, NEC, other disorders of nervous system in diseases classified elsewhere, hypertensive renal disease, subsequent myocardial infarction, cerebral infarction, cerebrovascular disorders in diseases classified elsewhere, sequelae of cerebrovascular disease, aortic aneurysm and dissection, stroke, not specified as haemorrhage or infarction, acute nephritic syndrome, chronic kidney disease, glomerular disorders in diseases classified elsewhere, faecal incontinence, abnormalities of gait and mobility, unspecified urinary incontinence, somnolence, stupor and coma, other symptoms and signs involving cognitive functions and awareness, other symptoms and signs involving general sensations and perceptions, symptoms and signs involving appearance and behavior, fracture of skull and facial bones, open wound of thorax, injury of other and unspecified intrathoracic organs, open wound of forearm, (5) fracture at wrist and hand level, fracture at wrist and hand level, and injury of muscle and tendon at hip and thigh level.

13. The method of claim 12, wherein the pre-processing sets such that, among the features,
total cholesterol is set to a value indicating that it is normal between 40 and 229 mg/dL, and is set to a value indicating that it is abnormal between 230 and 999 mg/dL,
hemoglobin is set to a value indicating that it is normal between 12 and 16.5 g/dL, and is set to a value indicating that it is abnormal between 0 g/dL and 12 g/dL, in the case of men, and is set to a value indicating that it is normal between 10 and 15.5 g/dL, and is set to a value indicating that it is abnormal between 0 g/dL and 10 g/dL, in the case of women,
serum GOT is set to a value indicating that it is normal between 0 and 50 U/L, and is set to a value indicating that it is abnormal between 51 and 999 U/L,
serum GPT is set to a value indicating that it is normal between 0 and 45 U/L, and is set to a value indicating that it is abnormal between 46 and 999 U/L, and
gamma GPT is set to a value indicating that it is normal between 11 and 77 U/L, and is set to a value indicating that it is abnormal between 78 and 999 U/L, in the case of men, and is set to a value indicating that it is normal between 8 and 45 U/L, and is set to a value indicating that it is abnormal between 46 and 999 U/L, in the case of women.

14. The method of claim 13, wherein the pre-processing sets features other than the total cholesterol, the hemoglobin, the serum GOT, the serum GPT, and the gamma GPT among the features to a value indicating one of the presence and absence of that disease.

15. A non-transitory recording medium readable by a computer system on which a program is recoded, wherein the program is for executing a method for processing data for predicting dementia through machine learning, wherein the method comprises:
pre-processing in which a value of each preset feature is set as a value to be input to the machine learning device based on the user's medical data; and
generating a data set including the value of each feature set by the pre-processing,
wherein each feature set in the pre-processing unit comprises at least one group of features of a first group of features, a second group of features, a third group of features, and a fourth group of features,
wherein the first group of features comprises at least one of hyperfunction of a pituitary gland, hypofunction and other disorders of the pituitary gland, other disorders of adrenal gland, and unspecified protein-energy malnutrition,
wherein the second group of features comprises at least one of calculus of lower urinary tract, urethral stricture, other disorders of male genital organs, inflammatory disease of uterus, except cervix, and polyp of female genital tract,
wherein the third group of features comprises at least one of kyphosis and lordosis, spinal osteochondrosis, and psoriatic and enteropathic arthropathies, and
wherein the fourth group of features comprises at least one of ascites, retention of urine, voice disturbances, malaise and fatigue, enlarged lymph nodes, and systemic inflammatory response syndrome.

16. The non-transitory recording medium of claim 15, wherein the user's medical data is received from a server managing the user's medical data through a communication network.

17. The non-transitory recording medium of claim 15, wherein the data set comprises the user's medical information for each year in the last 7 years or less.

18. The non-transitory recording medium of claim 15, wherein the preset feature further comprises hyperfunction of pituitary gland, hypofunction and other disorders of pituitary gland, other disorders of adrenal gland, unspecified protein-energy malnutrition, calculus of lower urinary tract, urethral stricture, other disorders of male genital organs, inflammatory disease of uterus, except cervix, polyp of female genital tract, kyphosis and lordosis, spinal osteochondrosis, psoriatic and enteropathic arthropathies, ascites, retention of urine, voice disturbances, malaise and fatigue, enlarged lymph nodes, and systemic inflammatory response syndrome.

19. The non-transitory recording medium of claim 15, wherein the preset feature further comprises total cholesterol, hemoglobin, serum GOT, serum GPT, gamma GTP, other disorders of pancreatic internal secretion, vitamin D deficiency, other disorders of thyroid, malnutrition-related diabetes mellitus, dementia in Alzheimer disease, vascular dementia, mental and behavioural disorders due to use of alcohol, acute and transient psychotic disorders, unspecified nonorganic psychosis, unspecified dementia, bipolar affective disorder, depressive episode, delirium, not induced by alcohol and other psychoactive substances, eating disorders, psychological and behavioural factors associated with disorders or diseases classified elsewhere, other mental disorders due to brain damage and dysfunction and to physical disease, schizophrenia, Parkinson disease, secondary parkinsonism, parkinsonism in diseases classified elsewhere, Alzheimer disease, other degenerative diseases of nervous system NEC, epilepsy, status epilepticus, transient cerebral ischemic attacks and related syndromes, vascular syndromes of brain in cerebro-vascular diseases, disorders of other cranial nerves, hemiplegia, paraplegia and tetraplegia, other paralytic syndromes, hydrocephalus, other disorders of brain, other disorders of nervous system, NEC, other disorders of nervous system in diseases classified elsewhere, hypertensive renal disease, subsequent myocardial infarction, cerebral infarction, cerebrovascular disorders in diseases classified elsewhere, sequelae of cerebrovascular disease, aortic aneurysm and dissection, stroke, not specified as haemorrhage or infarction, acute nephritic syndrome, chronic kidney disease, glomerular disorders in diseases classified elsewhere, faecal incontinence, abnormalities of gait and mobility, unspecified urinary incontinence, somnolence, stupor and coma, other symptoms and signs involving cognitive functions and awareness, other symptoms and signs involving general sensations and perceptions, symptoms and signs involving appearance and behavior, fracture of skull and facial bones, open wound of thorax, injury of other and unspecified intrathoracic organs, open wound of forearm, (5) fracture at wrist and hand level, fracture at wrist and hand level, and injury of muscle and tendon at hip and thigh level.

20. The non-transitory recording medium of claim 19, wherein the pre-processing sets such that, among the features,
total cholesterol is set to a value indicating that it is normal between 40 and 229 mg/dL, and is set to a value indicating that it is abnormal between 230 and 999 mg/dL,
hemoglobin is set to a value indicating that it is normal between 12 and 16.5 g/dL, and is set to a value indicating that it is abnormal between 0 g/dL and 12 g/dL, in the case of men, and is set to a value indicating that it is normal between 10 and 15.5 g/dL, and is set to a value indicating that it is abnormal between 0 g/dL and 10 g/dL, in the case of women,
serum GOT is set to a value indicating that it is normal between 0 and 50 U/L, and is set to a value indicating that it is abnormal between 51 and 999 U/L,
serum GPT is set to a value indicating that it is normal between 0 and 45 U/L, and is set to a value indicating that it is abnormal between 46 and 999 U/L, and
gamma GPT is set to a value indicating that it is normal between 11 and 77 U/L, and is set to a value indicating that it is abnormal between 78 and 999 U/L, in the case of men, and is set to a value indicating that it is normal between 8 and 45 U/L, and is set to a value indicating that it is abnormal between 46 and 999 U/L, in the case of women.
